(19) United States
(12) Patent Application Publication
(10) Pub. No.: US 2013/0325089 A1
(43) Pub. Date: Dec. 5, 2013

(54) METHOD AND APPARATUS FOR COOLING THE HEAD OR NECK OF A PATIENT

(71) Applicant: Regents of the University of Minnesota, St. Paul, MN (US)

(72) Inventors: Afshin A. Divani, Saint Paul, MN (US); Adam Gladen, Minneapolis, MN (US)

(73) Assignee: Regents of the University of Minnesota, St. Paul, MN (US)

(21) Appl. No.: 13/894,965

(22) Filed: May 15, 2013

Related U.S. Application Data
(60) Provisional application No. 61/647,058, filed on May 15, 2012.

(51) Int. Cl. A61F 7/00

(52) U.S. Cl. CPC A61F 7/0085
USPC 607/104

(57) ABSTRACT

A device for thermally coupling to a head and/or a neck of a patient is provided which includes cooling coils configured to allow for controlled cooling of the head or neck of the patient. The cooling coils are formed with tubing for directing cooling fluid. A plurality of fluid flow paths are provided whereby the flow of cooling fluid can be controlled to thereby selectively cool the head and/or neck of the patient.

### CROSS-REFERENCE TO RELATED APPLICATION

The present application is based on and claims the benefit of U.S. provisional patent application Ser. No. 61/647,058, filed May 15, 2012, the content of which is hereby incorporated by reference in its entirety.

### BACKGROUND

The present invention relates to the cooling of a patient. More specifically, the present invention relates to selective cooling of the head or neck region of the patient.

In recent years, much research has been conducted into the effects of inducing mild hypothermia in ischemic stroke and TBI (traumatic brain injury) patients. While it might seem apparent that mild hypothermia may be beneficial, only recently have pre-clinical and clinical studies been conducted to investigate its therapeutic effect with promising results.

The exact underlying effect of hypothermia as a neuro-protector is still under investigation. Studies indicate that hypothermia reduces cerebral metabolism of glucose, decreases brain oxygen consumption by approximately 5%/° C., and reduces inflammation and free radical generation. Inducing hypothermia may also help preserve neurons by reducing the neuronal damage from excitotoxins and apoptosis as well as inflammation. Hypothermia may also reduce the microcirculatory response to ischemia, minimizing granulocyte infiltration and microglial activation which could lead to a lower risk of post-reperfusion hemorrhage. This preservation can help to increase the time-window for other therapeutic approaches such as thrombolysis.

In rodents, studies have shown that hypothermia, thrombolysis, and hypothermia combined with thrombolysis improved the survival rates of the specimens with thromboembolic stroke, with the best results coming from hypothermia alone and hypothermia combined with thrombolysis. Even when the thrombolysis was delivered “late”, as long as hypothermia was administered, it improved the survival rates over even the “early” thrombolysis. This combination of hypothermia with reperfusion therapy is promising strategy in humans for transient ischemia.

The effects of hypothermia have also been investigated in other animal models as well, including gerbils, dogs, and mice. The trials have also shown that inducing hypothermia improves the survival rate for cerebral ischemia in these animal models. These trials also indicate the effectiveness of early application of hypothermia.

In addition to these animal trials, some limited human clinical trials have been performed. Two of the earliest and most notable studies were done in Europe by “The Hypothermia After Cardiac Arrest Study Group (HACA)” and in Australia by a group headed by Bernard et al. The HACA study looked at patients suffering from ventricular fibrillation cardiac arrest. The study showed that by cooling to 32-34 ° C., the survival rate was increased from 45% for the control group to 59% for the hypothermia group. The Australian group had similar out comes with 49% of the hypothermia group surviving with a good neurological outcome compared to 26% for the normothermia group.

A few limited studies of the effects of hypothermia in stroke patients have also been published. For example, long term hypothermia (48 to 72 hours) has been induced in patients with ischemic stroke. The neurological outcome was analyzed at 4 weeks and 3 months later, and it showed a reduction in mortality and an increase in the proportion of patients with a favorable outcome as compared with historical controls. Not all trials have proved to have conclusively positive results.

Thus, it has been shown that induced hypothermia can be therapeutically beneficial. However, the parameters related to the induction of hypothermia are also significant including the appropriate time-window, duration, and the target temperature for hypothermia to have the maximum therapeutic effect. In animal studies, a temperature range of 28-34° C. has produced the best results (although temperatures as low as 24° C. have been tested). In humans, the lowest temperature attempted (for a target temperature) has been 33° C. Lower temperatures in clinical setting may result in complications such as hypokalemia, arrhythmia, infections, hypothyroidism, and heart failure. Thus it appears that a cooling device should be able to achieve a target temperature of 33-35° C. in the core tissue. As to how long hypothermia should be applied, it seems to be dependent on when cooling is started. The sooner from the onset of ischemia that cooling can be administered, the more effective it will be. However, even if the start of cooling is delayed by a few hours, cooling may still have a neuro-protective effect if applied for a sufficiently long duration of time.

### SUMMARY

A device for thermally coupling to a head and/or a neck of a patient is provided which includes cooling coils configured to allow for controlled cooling of the head or neck of the patient. The cooling coils are formed with tubing for directing cooling fluid. A plurality of fluid flow paths are provided whereby the flow of cooling fluid can be controlled to thereby selectively cool the head and/or neck of the patient.

### BRIEF DESCRIPTION OF THE DRAWINGS

FIG. 1 is a diagram showing a cooling device in accordance with one example embodiment of the present invention.

FIG. 2 is a side profile view of a cooling garment shown in FIG. 1.

FIGS. 3A and 3B are a front and side view, respectively, of the cooling garment of FIG. 1.

FIG. 3B shows nasal cooling probes for use with the cooling device of FIG. 1.

FIG. 4 is a front open view of the cooling garment of FIG. 1.

FIG. 5 is a front view of the cooling garment of FIG. 1 illustrating positions of coils within the cooling garment.

FIG. 6 is a simplified schematic diagram of the cooling device of FIG. 1.

FIG. 7 is a schematic diagram showing another example configuration of a cooling device in accordance with the invention.

### DETAILED DESCRIPTION OF ILLUSTRATIVE EMBODIMENTS

As the apparent benefits to hypothermia have been explored and are beginning to be understood, various techniques can be used to induce that hypothermia. There are four primary methods to induce hypothermia:
Surface Cooling;
Cooled Saline Infusion;
Endovascular Heat Exchangers; and
A combination of cooling saline infusion with surface or endovascular cooling.

Each of these methods has benefits and shortcomings. Surface cooling has the advantage that it is the easiest to apply and the simplest. It does not require extremely advanced technology, and thus it can be done at lower cost. Further, it does not necessarily require knowledge of advanced treatment techniques such as catheter placement. Such cooling can also be applied in a pre-hospital setting thus allowing cooling to begin earlier. One drawback is that surface cooling is one of the slowest forms of cooling, and as the cooling is via the skin, it can induce shivering which (because it produces heat) will counter-act the cooling therapy. Surface cooling is also difficult to control and has a tendency to overshoot its intended target temperature. Moreover, surface cooling usually is done globally thereby cooling down the whole body rather than the affected area only.

Cooled saline infusions have shown promise in that it can cool the core temperature of a patient more rapidly than surface cooling and can simultaneously cool the internal body while heating techniques are applied to the skin to help combat shivering. One drawback is that relatively large volumes of infusion are required. Further, such cooling cannot be applied as precisely as surface cooling can be.

Endovascular heat exchangers are catheter based heat exchangers that are placed in the blood stream and can cool without an exchange of fluids. These, like the cooled saline infusion, allow cooling internally while warming the skin externally. They can also deliver cooling more precisely than the cooled saline since the cooling element remains in one place rather than traveling with the blood flow. Further, they also cool more rapidly than surface cooling. Historically though, endovascular heat exchangers have been associated with an increased risk of thrombosis, but this risk has been reduce recently with the advent of new coating materials that incorporate antithrombotics. These devices are the most technically challenging and complicated, and the most expensive. In addition, this solution requires knowledge of catheterization and endovascular surgery that can only be applied in a hospital with angiography equipment which limits its applications.

Returning to surface cooling, there are a few inherent challenges to induce local hypothermia in the head of a patient. The head has a low thermal conductivity, on the order of 0.5 W/° C.*m, and has a relatively large volume compared to the available surface area. Thus, one is attempting to cool an object that does not conduct heat very well and which does not provide much area for a device to contact. In addition to anatomical problems, there are the physiological issues that the brain produces heat through metabolic heat generation. This is fairly high compared to other organs. The brain is responsible for 15-20% of the body's resting metabolic heat production. Further, the perfusion of warm blood to the brain from the heart and core also makes cooling difficult. While the heat generation and perfusion are necessary for the proper brain tissue function, they make it challenging when attempting to induce hypothermia since being cooled is counter to the body's natural instinct.

These challenges are well documented in literature. This is explored, for example, in Nelson, D. A., & Nunneley, S. A. (1998). Brain temperature and limits on transcranial cooling in humans: Quantitative modeling results. European Journal of Applied Physiology, 78, 353. This numerical model looked at the effects of selective brain cooling. Nelson and Nunneley modeled the head as a hemisphere with different layers for the brain, cerebrospinal fluid (CSF), skull, and scalp with each layer having its own metabolic rate, physical properties, etc. Their results illustrate the difficulty of attempting to induce hypothermia beyond a superficial region of the brain. The edges of the brain closest to the skull cooled down but the central regions of the brain remain unaffected. As they stated, “the large size and low surface-to-volume ratio of the human cranium minimize the effect of superficial heat exchange regardless of the direction, volume or temperature of transcranial venous flow or the temperature of the face, scalp or respiratory mucosa.” Another important finding of the paper was that the primary determinant of brain temperature was the arterial temperature, thus highlighting the need to cool the blood flow coming into the brain.

A more comprehensive and anatomically correct finite-element model which looked at the same issue was done by Dennis, B. H., Eberhart, R. C., Dulikravich, G. S., & Radons, S. W. (2003). Finite-element simulation of cooling of realistic 3-D human head and neck. Journal of Biomechanical Engineering, 125, 832. In this model, rather than simplifying the geometry of the head to a sphere, a model was constructed that more closely mirrored that of a person and it was done in three dimensions. This study looked at such boundary conditions as ice packs applied directly to the head and the head being fitted with a cooling helmet. It was hoped that one of these boundary conditions would lead to an average brain temperature of 33° C. within 30 minutes of treatment beginning. The end result of their simulation indicated that neither method was able to obtain the desired goals. One of the major obstacles found to effective cooling was the warm blood perfusion throughout the brain.

However, there are some promising results for surface cooling. Strategically placed cooling pads (which circulated a cooled solution) have been used to induce mild hypothermia in a number of healthy individuals within two hours and maintained for three hours before the patients were slowly warmed back up to normal physiological temperatures. Zweifler, R. M., Voorhees, M. E., Mahmood, M. A., & Alday, D. D. (2003) Induction and Maintenance of Mild Hypothermia by Surface Cooling in Non-intubated Subjects. Journal of Stroke and Cerebrovascular Diseases, 12(5), 237. This was global body cooling and temperatures were monitored rectally and tympanically. In another study, over a longer period of time, local hypothermia was achieved in a limited number of healthy patients using a cooling element as illustrated in Radons, S. W., Nygaard, L. R., Abbenhouse, M. S., & Chester, S. M. In MedTronic Physio Control Corp. (Ed.), Rapid induction of mild hypothermia. US Patent 7179279. (607/108; 607/104; 607/109 ed.) A61F 7/00. In this study, hypothermia was induced and maintained for a period of 48 to 72 hours and the hypothermia was induced and maintained by the cooling helmet only (and not through full body measures such as ice baths). The core body temperature also reached into the mild hypothermia range, but remained a degree or two warmer than the brain temperature.

Various devices are available for inducing cooling in a patient. These include, for example, the RapidCool™ system available from MedCool, Inc. of Wellesley, Mass., systems available for the Mobile Ice system manufactured by Adroit Medical Systems of Loudon, Tenn., the Artic Sun system available from Medivance, Louisville, Colo., the CoolGuard3000® available from Zoll Medical Corporation of Chelmsford, Mass., the BeneChill cooling system available from Medtronic Inc., of Fridley, Minn. as well as systems available from TraumaTec of San Antonio, Tex..

The present invention provides an apparatus for inducing hypothermia by cooling the head and/or neck region of a patient. Various aspects include:
A sectional cooling abilities allowing independent cooling and control of different areas of the head and/or neck.
A closed-loop feedback system allowing independent monitoring and control of temperatures at desired target regions of the patient.
The ability to provide neck support to a patient, for example, to address spinal cord injuries.
Desired cooling abilities, for example, the ability to cool the brain at a depth of 2 inches by 1.5° C. with a desired time, i.e., 30-40 minutes.
Ease of use.
Ease of placement of the device on a patient.
Patient temperature measurement.

The present invention provides a method and apparatus for inducing local, mild hypothermia by cooling the head and/or neck of a patient. In one aspect, this is achieved by a cold fluid that is circulated through the device. The cold fluid can be carried in tubing arranged in a coil. The tubing can be divided up into zones, such that the area that is being cooled can be controlled. In one specific embodiment, the tubing forms a coil comprising tightly wrapped concentric circles of tubing which are banded together. In various embodiments, the device includes temperature sensors to provide feedback and may include a controller which is used to control flow of the cooling fluid through the tubing.

As discussed herein, the present invention includes a number of features and configurations. In one aspect, the present invention includes independent cooling regions which comprise two or more cooling zones that can be cooled independently from each other. This feature allows the temperature of different zones to be independently controlled. For example, the neck area can be cooled faster where the blood flow velocity is higher. Each of the independent cooling regions may optionally include a temperature feedback system to set or monitor a target temperature. In another configuration, the invention provides a head and/or neck cooling device with temperature monitoring probes situated proximate the skull, nasal cavity, oral cavity, neck, and/or ear cavity to provide feedback control and/or monitoring.

The device of the present invention may optionally include a neck brace to secure the neck of a patient in case of possible spinal cord injury applications. The neck brace itself may include optional embedded cooling channels. A cooling coil design can be provided, for example, circular or oval in shape, of tubing to allow for a reduction in the pressure head loss as the cooling fluid is circulated through the tubing. Further, this configuration provides a greater tubing to surface area ratio, thereby increasing the contact area and allowing for faster cooling. To provide greater heat transfer, the tubing itself may be of non-ferromagnetic metallic coils. Similarly, plastic tubing with embedded non-ferromagnetic metallic coils or non-ferromagnetic metallic powder may be employed to provide increased heat conductivity. The coils and head/neck cooling device may optionally be fabricated of material which is compatible with Magnetic Resonant Imaging (MRI) techniques.

In one configuration, the head and/or neck cooling device of the present invention includes cooling compartments (zones or regions) which are detachable. This allows access to the skull or neck of the patient for procedures such as ventricular catheter placement or intracranial pressure monitoring without the need to remove the cooling device from the patient.

An internal lining of the device may optionally be filled with a water soluble gel, other heat conducting material, adjustable air pressure, or the like. This allows the device to conform to the shape of the skull of the patient and thereby substantially eliminating any gaps between the cooling elements and the skull or neck of the patient.

A cooling reservoir may be employed in which multiple flow pumps are matched to various independent cooling zones of the device. This allows the flow rate through the various cooling zones to be independently controlled. A single pump may also be employed with adjustable valving to control the flow rate. The cooling fluid itself may be cooled using any appropriate technique. One example technique includes the use of a thermoelectric/peltier cooling device. In another example configuration, the cooling reservoir includes at least two separate insulated compartments. A thermoelectric/peltier cooling device may be positioned between the two compartments whereby heat is drawn from fluid in one compartment and transferred to fluid in the other compartment. In such a configuration, the fluid the cold compartment may be used to selectively cool the patient whereas the fluid in the warmer compartment may be used to selectively warm the patient. For example, the fluid in the cold compartment may be used for cooling the head and/or neck while the fluid in the warm compartment may be used to warm the hands or feet of the patient through warming gloves and socks to thereby reduce shivering during induced hypothermia. Other cooling techniques may also be used including the use of ice.

The cooling fluid may be any appropriate fluid. One example fluid is normal or hypertonic saline, or other appropriate fluid, to allow for a larger reduction in the temperature of the cooling fluid without causing the fluid to freeze.

The cooling device may optionally comprise cooling nasal prongs which are inserted into the nasal cavities of a patient. This configuration provides for the rapid cooling of the brain of the patient. A similar configuration uses a cooling flap or the like which is placed into the patient's mouth. This allows the direct cooling of the hard or soft pallet in the oral cavity of the patient.

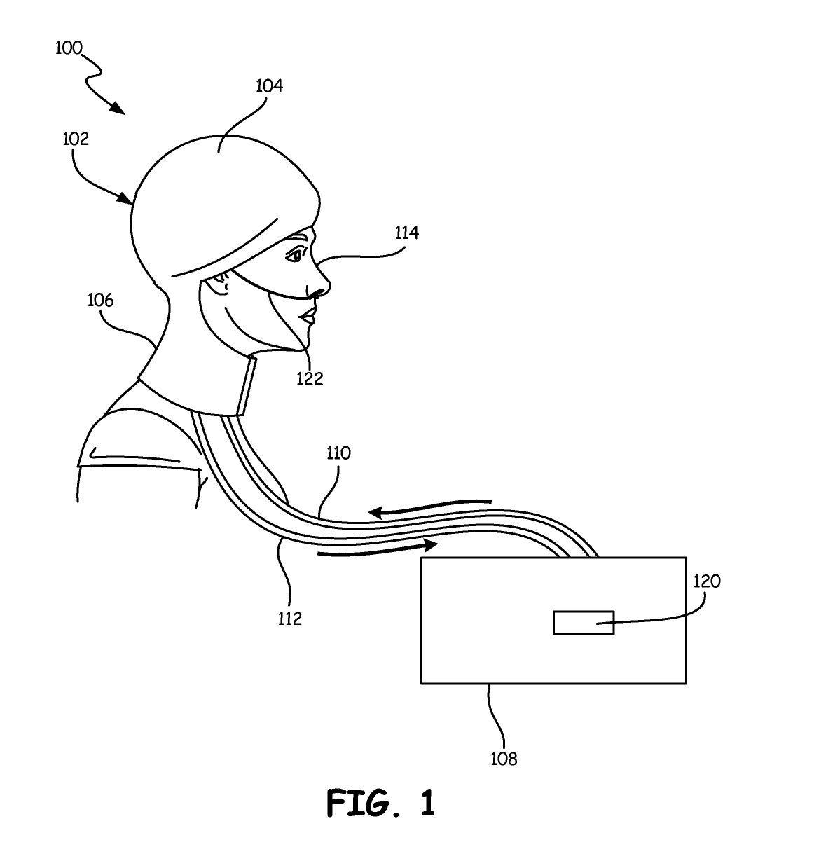

FIG. 1 is a diagram showing a cooling device 100 in accordance with one example embodiment of the present invention. As illustrated in FIG. 1, cooling device 100 includes a cooling garment 102 which may optionally comprise a head piece 104 and/or a neck piece 106. Cooling garment 102 is configured to be worn on the head and/or neck of a patient 114. Cooling fluid from a control system 108 which includes a cooling reservoir circulates through the cooling garment 104 to thereby cool the patient 114. In the example embodiment of FIG. 1, two tubes are used to connect the cooling garment 102 to the control system 108. A supply tube 110 supplies cooling fluid to the cooling garment and a return tube 112 returns the cooling fluid to the control system 108 after the cooling fluid has circulated through the cooling garment 102. An optional user interface 120 which, is described below in more detail, can be used to provide information to an operator and can be used by an operator to control operation of the cooling device 100. In some configurations, electrical connections are also provided which extend between cooling garment 106 and control system 108. The user interface 120 may be at any location, including positioned separately from the control system 108 and/or carried on the cooling garment 102.

FIG. 1 also illustrates nasal cooling connection 122 for use in coupling to nasal cooling prongs 124 (not shown in FIG. 1). The cooling prongs 124 can be used as standalone devices to cool the nasal passages of patient 114. Alternatively and as illustrated in FIG. 1, the nasal cooling prongs 124 can be used in conjunction with the cooling garment 102.

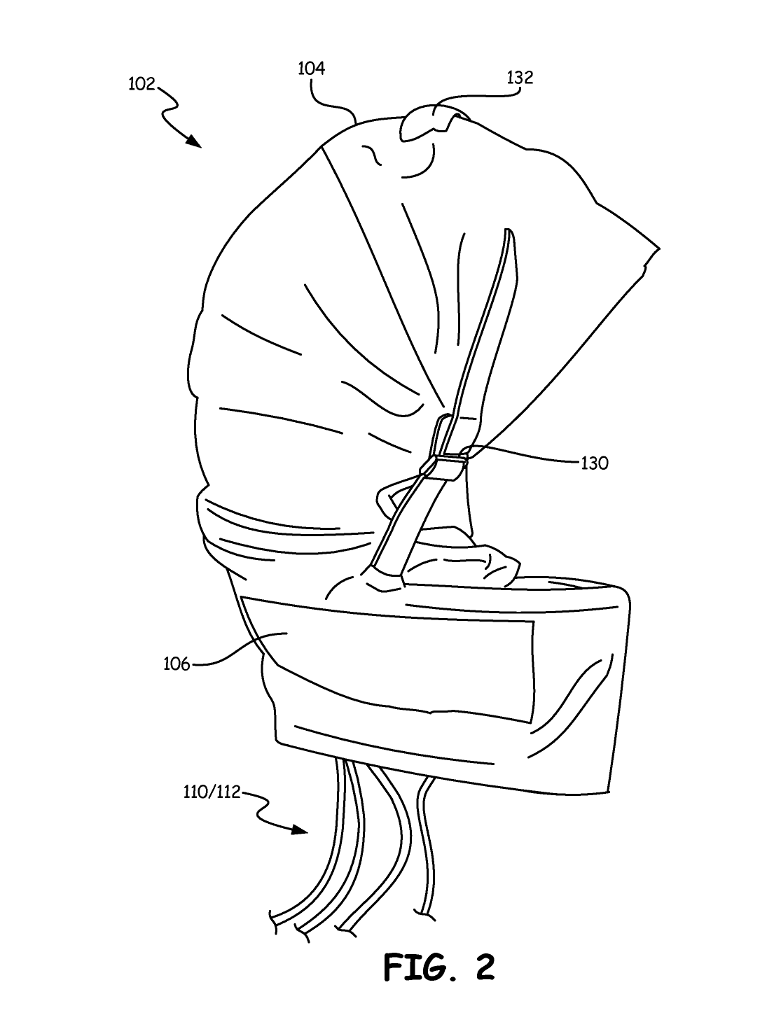

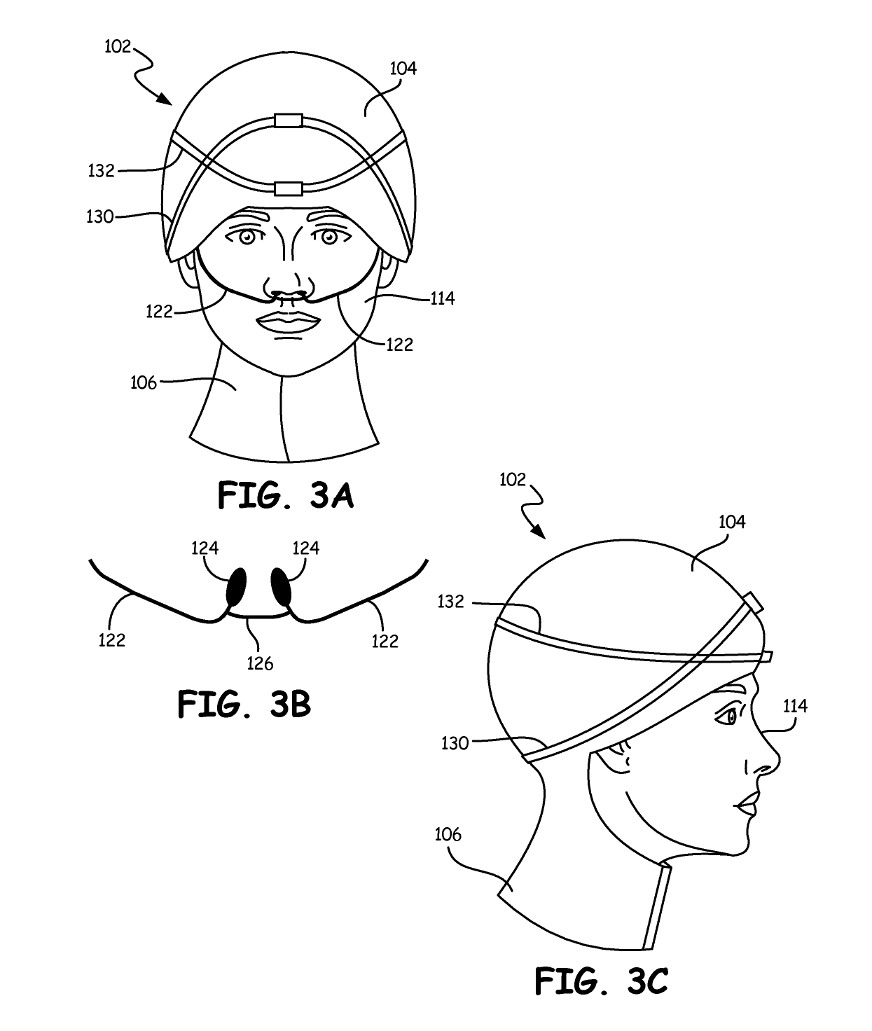

FIG. 2 is a side profile view of cooling garment 102. In the embodiment shown in FIG. 2, more than two cooling tubes 110/102 are used for carrying cooling fluid between control system 108 shown in FIG. 1 and the cooling garment 102. FIG. 2 also illustrates optional straps 130 and 132 used to secure the head piece 104 to the patient 114. A similar strap may be employed to secure the neck piece 106 to the neck of the patient 114. Although a buckle type connector is shown, any appropriate connector may be employed including, for example, hook and loop connectors. FIGS. 3A and 3B are front plan and side plan views, respectively, of another example configuration of cooling garment 102. FIG. 3B illustrates cooling connectors 122 coupled to nasal cooling prongs 124. Cooling connectors 122 carry cooling fluid to the prongs 124. For example, each cooling connector 122 can include two tubes, one for supplying cooling fluid and one for returning cooling fluid to the cooling fluid reservoir. In another example embodiment, a single cooling path is provided which extends from one cooling connector 122 to a prong 124, through a cooling bridge 126, through a second prong 124 and returning through a cooling connector 122.

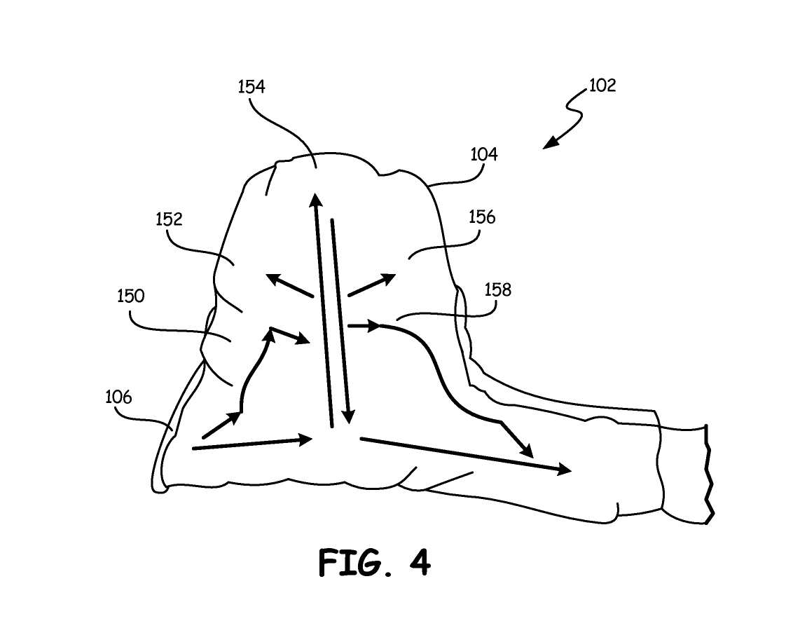

FIG. 4 is a front plan view of cooling garment 102 in which arrows illustrate the routing of tubing within the garment. In the configuration illustrated in FIG. 4, coils of tubing are positioned at five locations within the head piece 104 and form five different regions or zones thereby allowing selective cooling of the head of the patient 114. In FIG. 4, a right rear cooling region 150, a right front cooling region 152, a top cooling region 154, a left front cooling region 156, and a right rear cooling region 158 are illustrated. These five regions can be fed by five separate cooling tubes 110 from the control system 108 and the fluid returned to the control system 108 through a single return tube 112. In another embodiment, a single tube 110 is used and branches into regions 150-158.

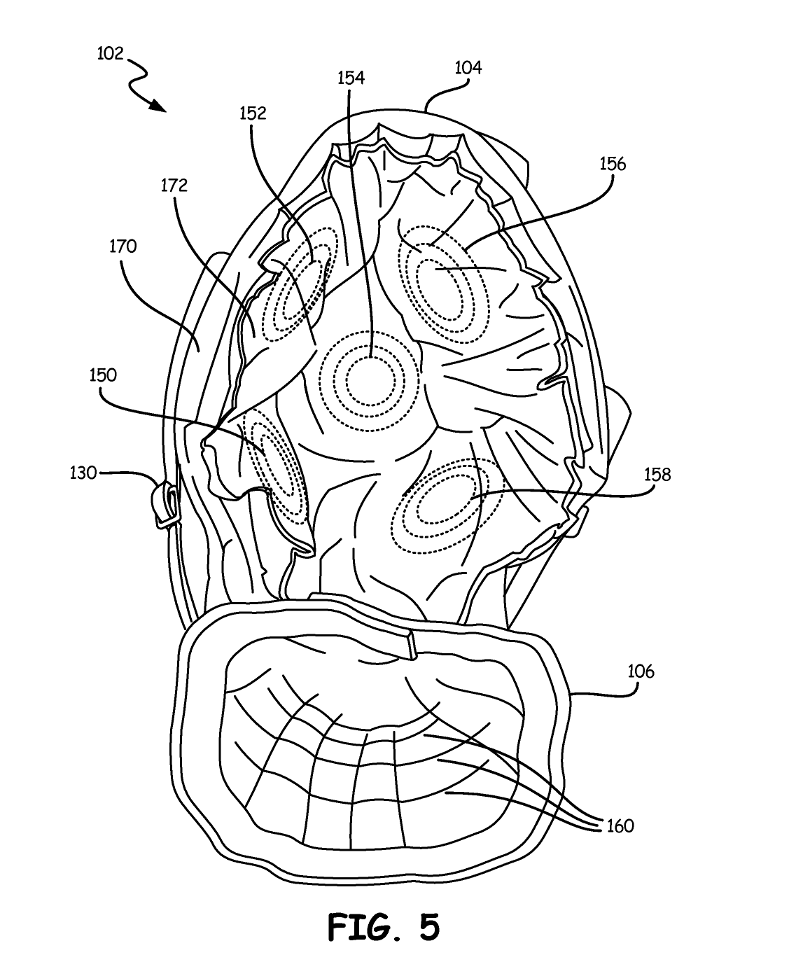

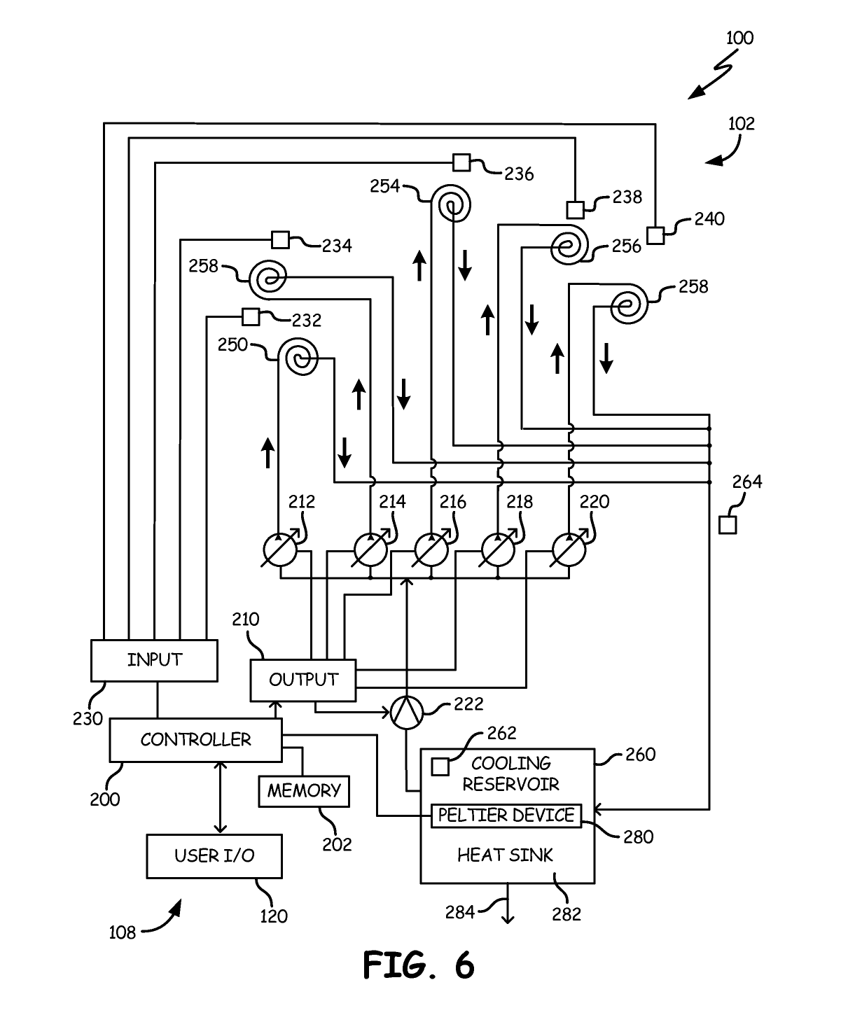

FIG. 5 is a perspective bottom view of cooling garment 102 and illustrates the position of cooling coils at regions 150, 152, 154, 156, and 158. In FIG. 6, tubing 160 is also illustrated in the neck piece 106 for use in cooling the neck of the patient 114. The various coils illustrated in FIG. 5 are held in position between an outer cover 170 and an inner liner 172 of the cooling garment 102. Additional padding or the like may be positioned between outer cover 170 and inner liner 172 for comfort and to assist in ensuring that the head piece 104 conforms to the head of the patient 114. In another example embodiment, the region between outer cover 170 and inner layer 172 is airtight whereby the space therebetween can be filled with pressurized air to assist in urging a greater surface area of the coils against the head and/or neck of the patient 114.

FIG. 6 is a simplified schematic diagram of cooling device 100 and shows the control system 108 in more detail. Control system 108 includes a controller 200 which may be a simple analog controller or, for example, may be more complex circuitry such as a digital controller which operates in accordance with programming instructions stored in a memory 202. One example digital controller is a microprocessor or the like. An optional user interface 120 can be used to control operation of controller 200. Output circuitry 210 is configured to control operation of valves 212, 214, 216, 218, and 220 as well as coolant pump 222. Input circuitry 230 is arranged to receive outputs from temperature sensors 232, 234, 236, 238, and 240. Sensors 232-240 are positioned proximate tubing coils 250, 252, 254, 256, and 258 which are located regions 150, 152, 154, 156, and 158, respectively, shown in FIG. 5.

During operation, controller 200 can be used to activate pump 222 which draws cooling fluid from a cooling reservoir 260. Controller 200 controls the amount of cooling fluid which is circulated through the coils by selectively adjusting the position of valves 212-220. Feedback may optionally be provided using temperature sensors 232-240. For example, the user input 120 may be used by an operator to set a desired temperature of a particular zone. By monitoring the temperature sensors 232-240, the controller 200 may selectively open or close valves 212-220 to obtain the desired temperature. For example, if the particular region is above a selected temperature, the appropriate valve may be opened further. Similarly, if a temperature is below a selected temperature, the appropriate valve may be closed. In another example configuration, elements 212-220 represent individual pumps which may be selectively controlled by controller 200 to thereby control the flow of cooling fluid into respective coils 250-258. Further, an optional temperature sensor 262 may be positioned in the cooling reservoir 260 to monitor the temperature of cooling fluid in the reservoir. The reservoir 260 may be cooled using any appropriate means. For example, ice may be used for passive cooling. Active techniques such as refrigeration techniques may also be employed. In the specific example illustrated, a Peltier device 280 is used and controlled by controller 200. During operation, the Peltier device 280 can be used to transfer heat from cooling reservoir 260 to a heat sink region 282. The transferred heat may be selectively removed using a heat output 284. For example, this heat output 284 can be used to warm portions of the patient such an extremities. Additional feedback may also be provided to controller 200, for example, using a temperature sensor 264 which monitors the temperature of the returning cooling fluid. The user interface 120 may be any type of interface including mechanical dials or buttons as well as more advance input such as keypads or other digital inputs. Optional outputs may also be included such as a display to provide temperature information, flow rate information, etc., and may be in an analog or digital format. Another example output may provide an indication that a temperature or flow rate has exceeded a threshold. For example, a light or other visual output may be turned on, or an audible output such as an alarm may be provided.

In another example configuration, one or more of the cooling coils 250-258 can be configured to be positioned proximate the hard or soft pallet of a patient, the neck of the patient using neck piece 106, the nasal cavity of the patient or other region of the head and/or neck of the patient. Such a configuration may also include additional coils for placement at other areas of the patient's head such as discussed above.

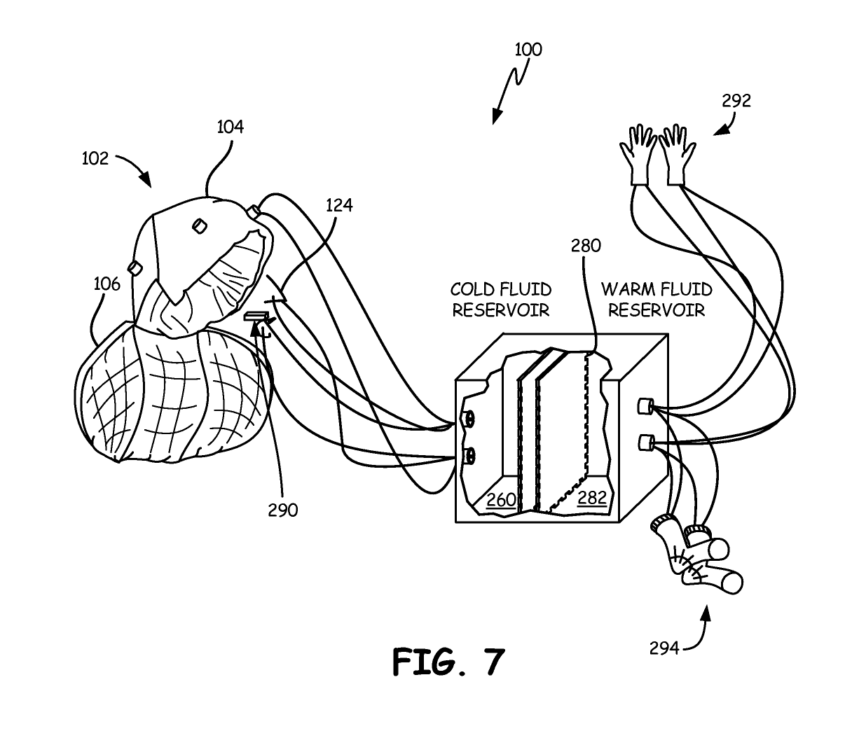

FIG. 7 is another example diagram of cooling device 100. In the configuration of FIG. 7, a pallet cooler 290 is provided for use in cooling a hard or soft pallet of the patient's mouth. FIG. 7 also illustrates warming garments 292 and 294 which can comprise, for example, gloves and socks, respectively. Warming garments 292 and 294 couple to warming reservoir (heat sink) 282 for use in warming the extremities of the patient as desired.

Although the present invention has been described with reference to preferred embodiments, workers skilled in the art will recognize that changes may be made in form and detail without departing from the spirit and scope of the invention.

What is claimed is:

1. An apparatus comprising:
a cooling garment configured to thermally couple to a head and/or a neck of a patient; and
cooling coils in the cooling garment configured to allow for controlled cooling of the head or neck of the patient.

2. The apparatus of claim 1, wherein the cooling garment comprises a head piece.

3. The apparatus of claim 1, wherein the cooling garment comprises a neck brace.

4. The apparatus of claim 1, wherein the cooling garment comprises a pallet cooler.

5. The apparatus of claim 1, wherein the cooling garment includes a plurality of strap adjusters removably fastened on a side of the cooling garment and is configured to secure the cooling garment to the head or the neck of the patient.

6. The apparatus of claim 1, wherein the cooling coils are removably connected to the head piece.

7. The apparatus of claim 1, wherein the cooling coils include metal.

8. The apparatus of claim 1, wherein the cooling coils comprise non-ferromagnetic metallic coils.

9. The apparatus of claim 1, including cooling nasal prongs configured to decrease temperature of nasal cavities of the patient.

10. The apparatus of claim 1, including a temperature sensor configured to monitor temperature of the patient.

11. The apparatus of claim 1, including internal lining removably attached to the cooling garment and placed between the cooling coils and the head piece.

12. The apparatus of claim 1, including a material capable of conducting heat positioned proximate the head and/or neck of the patient.

13. The apparatus of claim 1, wherein the cooling garment is pressurized to conform to the head and/or neck of the patient.

14. The apparatus of claim 1, including an opening in the cooling garment configured to provide access to a portion of the head or the neck of the patient.

15. The apparatus of claim 1 wherein the cooling coils define a plurality of independent cooling zones.

16. The apparatus of claim 15, wherein each of the plurality of independent cooling zones is configured to cool the head and/or the neck of the patient at a target temperature set by a user.

17. The apparatus of claim 1, including a cooling reservoir comprising two insulated compartments for use with a thermoelectric device configured to be positioned between the two insulated compartments.

18. A method for inducing hypothermia in desired regions of a human body, the method comprises:
placing a cooling garment on a head and/or a neck of a patient;
pumping a cooling fluid through a plurality of cooling coils in the cooling garment; and directing the cooling fluid to a plurality of independent cooling zones defined by the plurality of coils.

19. The method of claim 18, further comprising transferring heat from one side of a thermoelectric device to another side of the thermoelectric device for decreasing temperature of the cooling fluid.

20. The method of claim 19, further comprising sensing temperature at the plurality of cooling zones responsively controlling flow of cooling fluid through the plurality of cooling coils.
